(19) United States
(12) Patent Application Publication
(10) Pub. No.: US 2005/0226773 A1
(43) Pub. Date: Oct. 13, 2005

(54) Multiple modes acoustic wave sensor

(75) Inventor: James Z. Liu, Rockford, IL (US)

Correspondence Address: Kris T. Fredrick;Honeywell International Inc., 101 Columbia Rd., P.O. Box 2245, Morristown, NJ, 07952, US

(73) Assignee: Honeywell International, Inc.

(21) Appl. No.: 10/815,336

(22) Filed: Apr. 1, 2004

(51) Int. Cl. G01N 33/00

(52) U.S. Cl. 422/68.1

(57) ABSTRACT

A multiple mode sensing system is described, which can be configured from an acoustic wave sensor that includes a plurality of sensing components for monitoring a chemical species. The plurality of sensing components can be disposed within a cavity formed from a plurality of walls of said acoustic wave sensor, such that each sensing component is coated with a differing sensing film. The multiple modes sensing system also includes a plurality of oscillators associated with sensing components, wherein each sensing component is generally located in a feedback loop with identical oscillators to thereby provide a multiple modes acoustic wave sensor that provides multiple modes frequency outputs for the detection and desorption of a chemical species.

### TECHNICAL FIELD

Embodiments are generally related to sensing systems and methods. Embodiments are also related to acoustic wave sensors, such as, for example, surface acoustic wave (SAW) and bulk acoustic wave (BAW) devices and sensors.

### BACKGROUND OF THE INVENTION

Acoustic wave sensors are utilized in a number of sensing applications, such as, for example, temperature, pressure and/or gas sensing devices and systems. Examples of acoustic wave sensors include devices such as acoustic wave sensors, which can be utilized to detect the presence of substances, such as chemicals and biological materials. An acoustic wave (SAW/BAW) device acting as a sensor can provide a highly sensitive detection mechanism due to the high sensitivity to surface loading and the low noise, which results from their intrinsic high Q factor.

Surface acoustic wave devices are typically fabricated using photolithographic techniques with comb-like interdigital transducers placed on a piezoelectric material. Surface acoustic wave devices may have either a delay line or a resonator configuration. The selectivity of a surface acoustic wave chemical and/or biological sensor is generally determined by a selective coating placed on the piezoelectric material. The absorption and/or adsorption of the species to be measured into the selective coating can cause mass loading, elastic, and/or viscoelastic effects on the SAW/BAW device. The change of the acoustic property due to the absorption and/or adsorption of the species can be interpreted as a delay time shift for the delay line surface acoustic wave device or a frequency shift for the resonator (BAW/SAW) acoustic wave device.

Acoustic wave sensing devices often rely on the use of quartz crystal resonator components, such as the type adapted for use with electronic oscillators. In a typical gas-sensing application, the absorption of gas molecules in a selective thin film coating (i.e., applied to one surface of the crystal) can increase the mass of the crystal, while lowering the crystal's resonant frequency. The frequency of a thickness shear mode (TSM) crystal unit, for example, such as an AT-cut unit, is inversely proportional to the thickness of the crystal plate. For example, a typical 5-MHz 3rd overtone plate is on the order of 1 million atomic layers thick. The absorption of analyte is equivalent to the mass of one atomic layer of quartz, which changes the frequency by approximately 1 ppm.

The thickness-shear-mode resonators are therefore widely referred to as a quartz crystal microbalance. Calculations have determined that the sensitivity of a fundamental mode is approximately 9 times more sensitive than that of a 3rd overtone. A 5 MHz AT-cut TSM crystal blank, for example, is approximately 0.33 mm thick (fundamental). The thickness of the electrodes can be, for example, in a range of approximately 0.2-0.5 μm. The change in frequency due to the coating is typically: ΔF=−2.3×10⁶ F² (ΔM/A), where the value ΔF represents the change in frequency due to the coating (Hz), F represents the frequency of the quartz plate (Hz), ΔM represents the mass of deposited coating (g), and the value A represents the area coated (cm²).

Selective adsorbent thin film coated acoustic sensors such as, for example quartz crystal resonators, surface acoustic wave and quartz crystal microbalance devices are attractive to chemical/biological detection applications because of their high sensitivity, selectivity and ruggedness. The detection mechanism implemented depends on changes in the physicochemical and electrical properties of the coated piezoelectric crystal when exposed to gas. Measurement results are usually obtained as the output frequency of a loop oscillator circuit, which utilizes a coated crystal as the feedback element.

When the sensor is exposed to analytes, the thin film adsorbs the analytes, and a corresponding frequency shift is measured as a result of any physicochemical and electrical changes. Factors that contribute to the coating properties include coating density, coating modulus, substrate wetting, coating morphology, electrical conductivity, capacitance and permittivity. Coating materials selection, coating structures and coating techniques affect the sensors' responses.

Conventional techniques for thin film deposition vary extensively, depending on the properties of the coating materials and substrate. Examples of such techniques include CVD, PVD, and sol-gel for most of the inorganic and composite materials. For polymeric materials, self-assembly dipping methods, casting, spray coating, and/or spin coating from a solution of the polymer in a volatile solvent are often preferred. Configurations based on these conventional techniques generally determine the properties of an acoustic wave sensor. Coating methods are also important for a sensor's repeatability. Because of their relatively short lifetimes, such sensors are replaced more often than those based on metal oxide. When sensors are replaced, they lose their memory of previously learned odors. In other words, the response curves of such devices vary, and the replacement sensors must then be retrained and/or recalibrated.

For practical reasons, zeolites are widely utilized as the physisorption coating materials. Zeolites are crystalline alumino-silicates of alkali or alkaline earth elements (e.g., Li, Na, K, Mg, Ca, Ba) with frameworks based on extensive 3-dimentional networks of AlO₄ and SO₄ tetrahedra. These tetrahedra are assembled into secondary polyhedral building blocks such as cubes, octahedral and hexagonal prisms. The final zeolite structure consists of assemblages of the secondary blocks into a regular, 3-dimentional crystalline framework. Each aluminum atom has a (−1) charge and this gives rise to an anionic charge in the network.

Cations are necessary to balance the charge and occupy non-framework positions. Typically the framework is composed of a regular structure of interconnected cages and/or channels. These systems of essentially “empty” cages and/or channels provide the high storage capacities necessary for good adsorbents. Zeolite adsorbents are characterized by their uniform intra-crystalline aperture sizes. The uniformly sized apertures enable molecular discrimination on the basis of size (e.g., steric separation). Molecules larger than the maximum size that can diffuse into the crystal are excluded.

The sorption capacity and selectivity can be significantly affected by the type of cation used and the extent of ion exchange. This type of modification is important in optimizing zeolites for gas separation. The uniform pore structure, ease of aperture size modification, excellent thermal and hydrothermal stability, high sorption capacity at low partial pressures, and modest cost have made zeolites widely used in many separation application. For example, a selective adsorbent thin film coated quartz crystal microbalance chemical sensor can be utilized for the selective detection of CO. The thin coating comprises a solid non-porous inorganic matrix and porous zeolite crystals contained within the inorganic matrix, the pores of the zeolite crystals selectively adsorb chemical entities of a size less than a preselected magnitude.

The matrix can be selected from the group of sol-gel derived glasses, polymers and clay. The pores of the zeolite crystals are modified so as to be Lewis or Bronsted acidic or basic and capable of providing intrazeolite ligation by the presence of metal ions. The film can be configured from an alumina, boro-alumino-silicate, titanium, hydrolyzed diethoxydiphenyl silane, or silane rubber matrix containing zeolite crystals. The thickness of the inorganic matrix is generally about 0.001-10 μm and the diameter of the pores of the zeolite crystals is approximately 0.25-1.2 nm. The coating is a single layer of zeolite crystals protruding from an amorphous SiO₂ matrix.

A polymer can be defined as a compound consisting of a large number of repeating units, called monomers. These monomers are joined together by covalent bonds to form a long chain. The degree of polymerization is defined as the number of repeating units in the chain. The properties of the polymer depend on the overall size of the polymer chain and on the inter- and intra-molecular forces that hold the polymer together. In general, the polymer properties of interest can be characterized as diffusion/permeation properties or as mechanical properties. The measurement of diffusion/permeation properties is straightforward when diffusion of a species into a polymer film produces a simple mass-loading effect. Polymers used as sensor coatings are butyl rubber, cellulose polymers, polysiloxanes, polyaniline and polyethylene, and the like.

Polymers, specifically rubbery, amorphous polymers, have several inherent advantages as chemically sensitive sensor coatings. They can be deposited as thin, adherent, continuous films of fairly uniform thickness by solvent casting or spray coating. They are nonvolatile and of homogeneous composition, and their chemical and physical properties can be modified to some extent by judicious choice of monomers and synthetic procedures. The glass transition temperature Tg, is the temperature at which a polymer changes from glassy to rubbery. Above Tg, permeability is governed entirely by diffusion forces and adsorption proceeds rapidly and reversibly. One more advantage of rubbery, amorphous polymers is that their sorption isotherms are often linear over relatively large ranges in penetrant concentration.

In general, the coated adsorbent thin film must be uniform, adherent, thin, chemically and physically stable when in contact with its working medium. Uniformity in film thickness is not crucial, but can be important in some cases, i.e., when the rate of permeation is used to identify an analyte. The selectivity of the acoustic wave sensor is influenced by the structure of the coatings. The different film structures and thus different response properties can be achieved by varying the ratio of the materials forming the sensing film.

In order to construct a sensing film with desired response properties, the analyte molecules and sensing film materials can be mixed in a solution which in order to result in the most suitable formation because of affinity. The interaction force is selected by the affinity between the sensing film and analyte. This can easily result in a sensor with desired response properties. In the case of a gas sensor, in order to achieve the same result, one should fabricate the adsorbent thin film in a glove box filled with the sample gas. Other methods include molecular imprinting (i.e., forming specific sorption sites using molecularly imprinted polymers) and host-guest interaction (i.e., a result of structural interaction between a host molecule, such as cyclodextrin, and a guest molecule).

Acoustic wave sensors, which are coated with affinity/adsorption type sensing materials thus may possess problems when desorbing the analyte(s) after the sensor is exposed to the analyte(s), thereby increasing the response time and running the risk of losing functionalities following the initial exposure of the sensor to the substance sought to be detected by the sensor. A need thus exists for an improved acoustic wave sensor, which can overcome these problems, and particularly, one which does not result in response time increases and the loss of functionalities following the initial exposure of the sensor to the substance sought to be detected. It is believed that sensor disclosed herein overcomes these problems.

### BRIEF SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

It is, therefore, one aspect of the present invention to provide improved sensor methods and systems.

It is another aspect of the present invention to provide for improved acoustic wave sensor methods and systems.

It is yet a further aspect of the present invention to provide an improved multiple mode surface acoustic wave (SAW) or bulk acoustic wave (BAW) sensing system.

It is also an aspect of the present invention to dual mode surface acoustic wave (SAW) sensing system.

The aforementioned aspects of the invention and other objectives and advantages can now be achieved as described herein. A multiple mode sensing system is described herein, which can be configured from an acoustic wave sensor comprising a plurality of sensing components for monitoring a chemical species. The plurality of sensing components can be disposed within a cavity formed from a plurality of walls of said acoustic wave sensor, such that each sensing component of said plurality of sensing components is coated with a differing sensing film. The multiple mode sensing system also includes a plurality of oscillators associated with said plurality of sensing components, wherein each sensing components of said plurality of sensing components is generally located in a feedback loop with an oscillator of said plurality of oscillators to thereby provide a multiple mode acoustic wave sensor that provides multiple mode frequency outputs thereof, wherein a calculated difference among said multiple mode frequency outputs is utilized to promote an increase in sensing accuracy by eliminating responses due to environmental changes other than said monitored chemical species.

Each sensing component can comprise a quartz crystal. The multiple mode frequency outputs can comprise one or more of the following types of data: flexural plate mode (FMP) data, acoustic plate mode data, shear-horizontal acoustic plate mode (SH-APM) data, amplitude plate mode (APM) data, thickness shear mode (TSM) data, surface acoustic wave mode (SAW), bulk acoustic wave mode (BAW) data, torsional mode data, love wave data, leaky surface acoustic wave mode (LSAW) data, pseudo surface acoustic wave mode (PSAW) data, transverse mode data, surface-skimming mode data, surface transverse mode data, harmonic mode data, and overtone mode data.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a block diagram of a multiple mode acoustic wave sensor system, which can be implemented in accordance with a preferred embodiment of the present invention;

FIG. 2 illustrates a block diagram of a multiple mode acoustic wave sensor system, which can be implemented in accordance with an alternative embodiment of the present invention;

FIG. 3 illustrates a diagram depicting varying modes, which can be utilized for desorption in affinity/adsorption type sensors, in accordance with preferred or alternative embodiments of the present invention; and

FIG. 4 illustrates a high-level flow chart of operations depicting logical operational steps, which can be implemented in accordance with a preferred embodiment of the present invention.

### DETAILED DESCRIPTION OF THE INVENTION

The particular values and configurations discussed in these non-limiting examples can be varied and are cited merely to illustrate at least one embodiment of the present invention and are not intended to limit the scope of the invention.

Many modes of vibrations can exist in an acoustic wave device. Acoustic wave devices are typically designed such that only one mode of vibration is optimized, while other modes are suppressed. Such “undesired” mode(s), however, can be utilized for desorption in affinity/adsorption type sensors. Such modes can include, for example, flexural plate mode (FPM), acoustic plate mode, shear-horizontal acoustic plate mode (SH-APM), amplitude plate mode (APM), thickness shear mode (TSM), surface acoustic wave mode (SAW), bulk acoustic wave mode (BAW), Torsional mode, love wave, leaky surface acoustic wave mode (LSAW), pseudo surface acoustic wave mode (PSAW), transverse mode, surface-skimming mode, surface transverse mode, harmonic modes, and/or overtone modes. Thus, in accordance with embodiments disclosed herein, multiple vibration modes can be utilized to produce a multiple mode acoustic wave device.

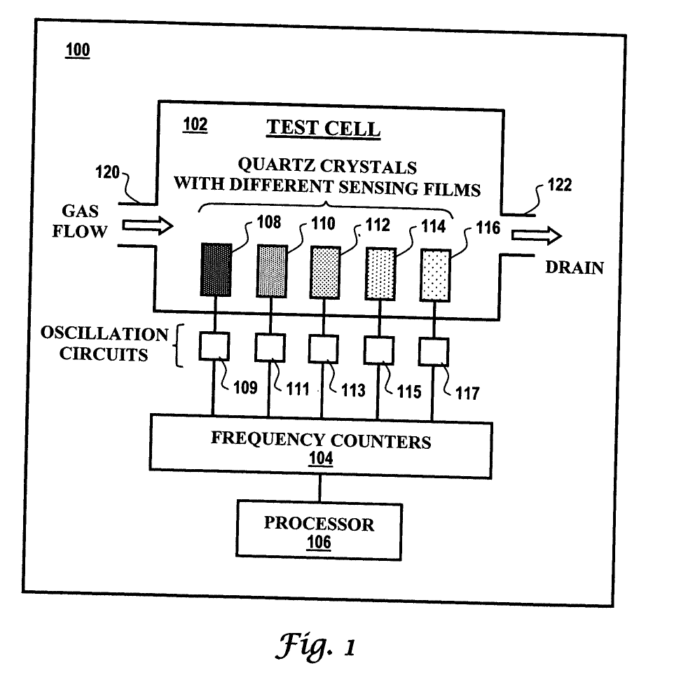

FIG. 1 illustrates a block diagram of an acoustic wave sensor system 100, in which an embodiment of the present invention can be implemented. System 100 can be implemented as an array of sensors, such as, for example, a plurality of quartz crystals 108, 110, 112, 114, and 116, which are located within a test cell 102. Each quartz crystal can be placed in a feedback path of an oscillator. For example, quartz crystal 108 can be placed in a feedback path of oscillator circuit 109, while quartz crystal 110 is generally placed in the feedback path of oscillator circuit 111. Similarly, quartz crystal 112 can be place in the feedback path of oscillator circuit 113, while quartz crystal 113 is generally placed in the feedback path of oscillator circuit 115. Finally, quartz crystal 116 is generally placed in the feedback path of oscillator circuit 117. Oscillator circuits 109, 111, 113, 115 and 117 communicate with frequency counter 104, which in turn is under the command of a processor 106. In practice, gas flow or another chemical flow can enter test cell 102 through an entrance 120 and exist via a drain 122.

In terms of coating selection for an array of sensors, such as system 100 depicted in FIG. 1, a minimum number of sensor/coatings can be implemented, in order to adequately represent the data. Thus, coatings exhibiting similar or redundant response should be eliminated. A coating, when selected from a group of coatings, should be based on considerations such as sensitivity, stability or cost.

The selectivity of a chemical gas sensor can be improved by taking advantage of selective adsorbent materials. Some improvement can be achieved by utilizing selective permeable filters. Interferences, however, may not always be known before the use of sensor. In addition, applications that require simultaneous monitoring for multiple analytes require multiple sensors. In such cases, the use of arrays of sensors, each bearing a coating with a different degree of selectivity for the analytes of interest, can be utilized.

In terms of pattern-recognition analysis, a coating can be classified according to its response to a set of analytes. Each sensor in an array can be designed with a different coating, wherein each coating is selected to respond differently to the members of a set of analytes. The combination of responses should produce a unique fingerprint for each analyte. A number of methods have been developed for establishing correlations between the pattern of responses from an array of chemical sensors and identity of the corresponding analyte. The efficiency of the array depends on the uniqueness of the coating responses.

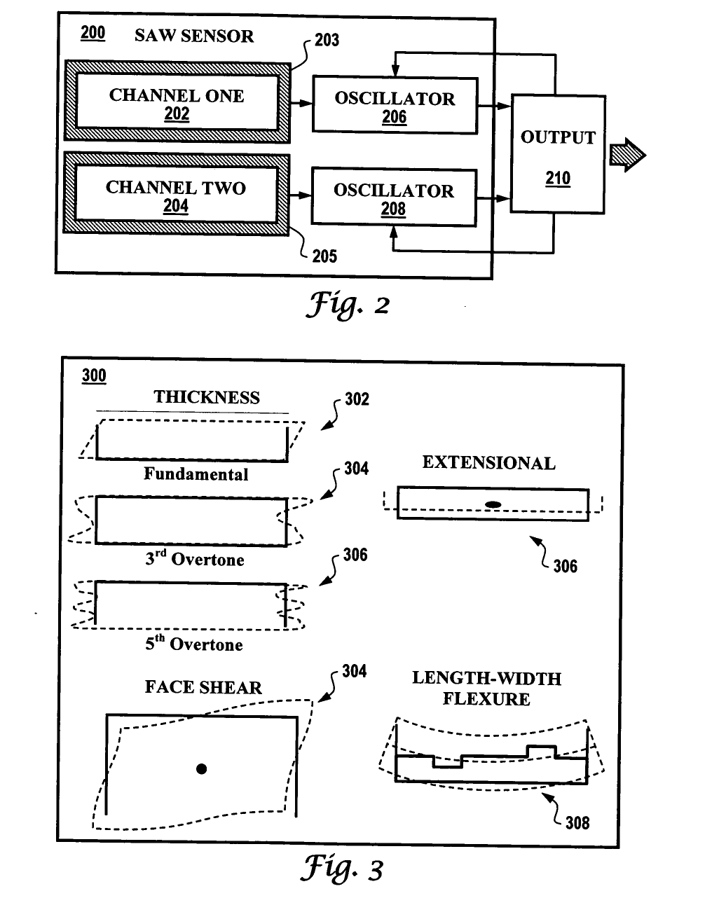

FIG. 2 illustrates a block diagram of a multiple mode acoustic wave sensor system 200, which can be implemented in accordance with an alternative embodiment of the present invention. System 200 can be implemented as a two-channel SAW sensor, composed of a first channel 202 and a second channel 204. First channel 202 is composed of a sensing coating 203, while second channel 204 includes a sensing coating 205. Each channel 202 and 204 can include a quartz crystal sensing component. Second channel 204 includes a quartz crystal structure, which is identical to that contained by first channel 202, except for the sensing coatings 203 and 205. The two channels 202 and 204 can be placed in the feedback path of two identical oscillators 206 and 208, and the output 210 of the circuit is the difference of the two frequencies produced thereof. With this arrangement, the SAW sensor system 200 can increase the sensing accuracy by eliminating response due to changes in the environment other than the monitored chemical species.

Selective adsorbent coating materials can be used for different gaseous detection applications. Examples of such coating materials include NO₂, SO₂, CO₂, H₂S, NH₃, HCl, water vapor and hydrocarbons. Adsorption occurs due to molecular interactions between the adsorbing species and the solid. Chemisorptions occur when strong interactions, including hydrogen bonding and covalent and ionic bond formation. Chemisorptions occur even at very low concentrations, and the chemisorption species are often “irreversible” bound to the surface, i.e., they will not readily desorb under ambient temperature conditions. In accordance with another embodiment of the present invention, however, SAW sensor system 200 can be implemented as a SAW/BAW humidity/dew point sensor. While humidity sensors tend to condense at the surface of sensing materials. The use of multiple modes can therefore shake away the water droplet and the sensor will recover quickly from water saturation.

Frequency can be measured with far higher accuracy than any other quantity. Dual modes excitation such as that provided by system 200 can provide superior sensing because the two modes occupy the same volume of quartz. In multiple modes excitation, the multiple excited modes occupy the same volume of piezoelectric material. Multiple modes can be excited simultaneously by means of multiple oscillator circuits sharing a common piezoelectric device. In this design, however, other modes are designed to be excited after the sensor's exposure to the analyte(s)

The piezoelectric substrate materials could be α-quartz, lithium niobate (LiNbO3), and lithium tantalate (LiTaO3) as well as Li2B4O7, AlPO4, GaPO4, langasite (La3Ga5SiO14), ZnO, and epitaxially grown (Al, Ga, In) nitrides. The electrode material for the piezoelectric device could be divided into three groups: metals (e.g. Al, Pt, Au, Rh, Ir, Cu, Ti, W, Cr, Ni), alloys (e.g. NiCr, CuAl) and metal-nonmetal compounds (e.g. ceramic electrodes: TiN, CoSi2, WC).

Selective adsorbent coating materials have been used for different gas/chemical/biochemical materials detections. Adsorption occurs due to molecular interactions between the adsorbing species and the solid. Chemisorption occurs when strong interactions, including hydrogen bonding and covalent and ionic bond formation. Chemisorption occurs even at very low concentrations, and the chemisorption species are often “irreversibly” bound to the surface. In other words, they will not readily desorb under ambient temperature conditions.

Physical adsorption represents a weak interaction, typically van der Waals forces. Common materials for physical sorption can include, for example, activated charcoal, silica and alumina gels, zeolites, porous polymers (e.g., Tenax, XAD, Chromosorb). Adsorbents tend to be micro-porous solids possessing large surface areas (e.g., 200 to 1000 m²/g). A high degree of discrimination is achieved by the use of size specific materials, having a controlled pore size just larger than the kinetic diameter of the desired analyte. This excludes all larger species from the pores entirely; molecules significantly smaller than the chosen analyte, though able to fit into the pores, have a smaller interaction energy due to the size mismatch.

Vibrations of acoustic wave devices could be used to break down the bonding (i.e., connections) between the analytes(s) and the sensing materials. A variety of acoustic modes may propagate in a piezoelectric device, this includes bulk waves and surface waves. For most acoustic wave devices the substrate materials and crystal orientation are usually chosen such that the only one mode that can be excited. However, other modes could be excited. The vibrational frequencies and amplitudes can be chosen, such that they are most suitable for breaking the bonding between the sensing materials and analyte(s).

FIG. 3 illustrates a diagram depicting varying modes 300, which can be utilized for desorption in affinity/adsorption type sensors, in accordance with preferred or alternative embodiments of the present invention. For example, “thickness” is depicted in FIG. 3, including a fundamental 302, 3^{rd} overtone 304 and 5^{th} overtone 306. A face shear 304 is also depicted in FIG. 3, along with an extensional 306 and a length-width flexure 308. FIG. 3 illustrates the fact that many modes of vibrations can exist in an acoustic wave device, and that acoustic wave and/or BAW devices are typically designed such that only one mode of vibration is optimized, while other modes are suppressed. According to the embodiments described herein, such “undesired” mode(s), can be utilized for desorption in affinity/adsorption type sensors. Such modes can include, for example, flexural plate mode (FPM) (e.g., see length-width flexure 308), shear-horizontal acoustic plate mode (SH-APM) (e.g., see face shear 304), and thickness shear mode (TSM) (e.g., see fundamental 302, 3^{rd} overtone 304 and 5^{th} overtone 306). It can be appreciated of course that such modes are only a few of many other types of modes which can be utilized in accordance with preferred or alternative embodiments, and are referred to herein for illustrative purposes only.

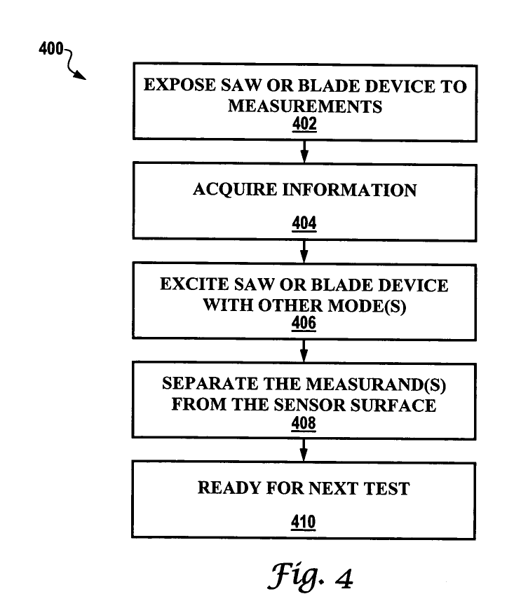

FIG. 4 illustrates a high-level flow chart 400 of operations depicting logical operational steps, which can be implemented in accordance with a preferred embodiment of the present invention. As indicated at block 402, the SAW or BAW sensor device can be exposed to various modal measurements, as described herein. Thereafter, as depicted at block 404, such modal information can be acquired. Next, as illustrated at block 406, the SAW or BAW device can be excited with one or more other modes. Thereafter, as illustrated at block 408, the measurand(s) can be separated from the sensor surface. Finally, as depicted at block 410, the sensor is ready for the next test.

The embodiments and examples set forth herein are presented to best explain the present invention and its practical application and to thereby enable those skilled in the art to make and utilize the invention. Those skilled in the art, however, will recognize that the foregoing description and examples have been presented for the purpose of illustration and example only. Other variations and modifications of the present invention will be apparent to those of skill in the art, and it is the intent of the appended claims that such variations and modifications be covered.

The description as set forth is not intended to be exhaustive or to limit the scope of the invention. Many modifications and variations are possible in light of the above teaching without departing from the scope of the following claims. It is contemplated that the use of the present invention can involve components having different characteristics. It is intended that the scope of the present invention be defined by the claims appended hereto, giving full cognizance to equivalents in all respects.

1. A multiple modes sensing system, comprising:
an acoustic wave sensor comprising a plurality of sensing components for monitoring a chemical species, wherein said plurality of sensing components is disposed within a cavity formed from a plurality of walls of said acoustic wave sensor, wherein each sensing component of said plurality of sensing components is coated with a differing sensing film; and
a plurality of oscillators associated with said plurality of sensing components, wherein each sensing components of said plurality of sensing components is located in a feedback loop with an oscillator of said plurality of oscillators to thereby provide a multiple mode acoustic wave sensor that provides multiple mode frequency outputs thereof, wherein a calculated difference among said multiple mode frequency outputs is utilized to promote an increase in sensing accuracy by eliminating responses due to environmental changes other than said monitored chemical species.

2. The system of claim 1 wherein each sensing component of said plurality of sensing components comprises a quartz crystal.

3. The system of claim 1 wherein said multiple modes frequency outputs comprise at least one of the following types of data: flexural plate mode (FMP) data, acoustic plate mode data, and shear-horizontal acoustic plate mode (SH-APM) data.

4. The system of claim 3 wherein said multiple mode frequency outputs further comprises at least one of the following types of data: amplitude plate mode (APM) data, thickness shear mode (TSM) data, surface acoustic wave mode (SAW), and bulk acoustic wave mode (BAW) data.

5. The system of claim 4 wherein said multiple mode frequency outputs further comprises at least one of the following types of data: torsional mode data, love wave data, leaky surface acoustic wave mode (LSAW) data, and pseudo surface acoustic wave mode (PSAW) data, and at least one multiple mode acoustical vibration amplitude.

6. The system of claim 5 wherein said multiple mode frequency outputs further comprises at least one of the following types of data: transverse mode data, surface-skimming mode data, surface transverse mode data, harmonic mode data, and overtone mode data.

7. The system of claim 5 wherein said at least one multiple mode acoustical vibration amplitude is controlled by said plurality of oscillators.

8. The system of claim 7 wherein said at least one multiple mode acoustical vibration amplitude is controlled by said plurality of oscillators in order to produce vibrations for shaking away analytes from bonding surfaces thereof.

9. The system of claim 1 said acoustic wave sensor comprises a SAW sensor.

10. The system of claim 9 wherein said SAW sensor comprises a humidity sensor, which provides multiple modes that shake away any water droplets condensing upon said SAW sensor, thereby permitting said SAW sensor to recover quickly from water saturation.

11. The system of claim 1 wherein said multiple mode acoustic wave sensor produces vibrations which break down bonding connections between analytes and said plurality of sensing components.

12. The system of claim 1 wherein said sensing components of said plurality of sensing components comprise electrode materials chosen from among a group comprising at least one of the following metals: Al, Pt, Au, Rh, Ir, Cu, Ti, W, Cr, and Ni.

13. The system of claim 1 wherein said sensing components of said plurality of sensing components comprise electrode materials chosen from among a group comprising at least one of the following alloys: TiN, CoSi2, and WC.

14. The system of claim 1 wherein said sensing components of said plurality of sensing components comprise electrode materials chosen from among a group comprising at least one of the following metal-nonmetal compounds: NiCr and CuAl.

15. A dual modes sensing system, comprising:
an acoustic wave sensor comprising two sensing components for monitoring a chemical species, wherein each sensing component are disposed within a respective channel within a cavity formed from a plurality of walls of said acoustic wave sensor, such that each of said sensing components is coated with a differing sensing film; and
two identical oscillators associated with said sensing components, wherein each of said sensing components is located in a feedback loop with each of said two identical oscillators to thereby provide a dual mode acoustic wave sensor that provides dual mode frequency outputs thereof, wherein a calculated difference among said dual mode frequency outputs is utilized to promote an increase in sensing accuracy by eliminating responses due to environmental changes other than said monitored chemical species.

16. The system of claim 15 wherein each sensing component comprises a quartz crystal.

17. The system of claim 15 wherein said dual mode acoustic wave sensor produces vibrations which break down bonding connections between analytes and said plurality of sensing components.

18. The system of claim 15 wherein said sensing components comprise piezoelectric materials chosen from among a group comprising at least one of the following materials: α-quartz, lithium niobate (LiNbO3), lithium tantalate (LiTaO3), Li2B4O7, AlPO4, GaPO4, langasite (La3Ga5SiO14), ZnO, and epitaxially grown nitrides including Al, Ga or In.

19. A multiple modes sensing system, comprising:
a surface acoustic wave (SAW) sensor comprising a plurality of quartz crystal sensing components for monitoring a chemical species, wherein said plurality of quartz crystal sensing components is disposed within a cavity formed from a plurality of walls of said acoustic wave sensor, wherein each quartz crystal sensing component of said plurality of quartz crystal sensing components is coated with a differing sensing film; and
a plurality of oscillators associated with said plurality of quartz crystal sensing components, wherein each quartz crystal sensing components of said plurality of quartz crystal sensing components is located in a feedback loop with an oscillator of said plurality of oscillators to thereby provide a multiple mode SAW sensor that provides multiple mode frequency outputs thereof, wherein a calculated difference among said multiple mode frequency outputs is utilized to promote an increase in sensing accuracy by eliminating responses due to environmental changes other than said monitored chemical species.

20. The system of claim 19 wherein said multiple mode SAW sensor comprises a humidity sensor, which provides multiple modes that shake away any water droplets condensing upon said multiple mode SAW sensor, thereby permitting said multiple mode SAW sensor to recover quickly from water saturation; and
wherein said multiple modes SAW sensor produces vibrations which break down bonding connections between analytes and said plurality of quartz crystal sensing components.
